SEQUENCE LISTING (14 sequences)

SEQ ID NO: 1 - DNA, 1863 bp, Homo sapiens
aggagccgccgccagtggagggccgggcgctgcggccgcggccggggcgggcgcagggccgagcggacgggggggcgcgggccccccgggaggccgcggccactcccccccgggccggcgcggcgggggaggcggaggatggaaacacccttctacggcgatgaggcgctgagcggcctgggcggcggcgccagtggcagcggcggcagcttcgcgtccccgggccgcttgttccccggggcgcccccgacggccgcggccggcagcatgatgaagaaggacgcgctgacgctgagcctgagtgagcaggtggcggcagcgctcaagcctgcggccgcgccgcctcctacccccctgcgcgccgacggcgcccccagcgcggcaccccccgacggcctgctcgcctctcccgacctggggctgctgaagctggcctcccccgagctcgagcgcctcatcatccagtccaacgggctggtcaccaccacgccgacgagctcacagttcctctaccccaaggtggcggccagcgaggagcaggagttcgccgagggcttcgtcaaggccctggaggatttacacaagcagaaccagctcggcgcgggcgcggccgctgccgccgccgccgccgccgccggggggccctcgggcacggccacgggctccgcgccccccggcgagctggccccggcggcggccgcgcccgaagcgcctgtctacgcgaacctgagcagctacgcgggcggcgccgggggcgcggggggcgccgcgacggtcgccttcgctgccgaacctgtgcccttcccgccgccgccacccccaggcgcgttggggccgccgcgcctggctgcgctcaaggacgagccacagacggtgcccgacgtgccgagcttcggcgagagcccgccgttgtcgcccatcgacatggacacgcaggagcgcatcaaggcggagcgcaagcggctgcgcaaccgcatcgccgcctccaagtgccgcaagcgcaagctggagcgcatctcgcgcctggaagagaaagtgaagaccctcaagagtcagaacacggagctggcgtccacggcgagcctgctgcgcgagcaggtggcgcagctcaagcagaaagtcctcagccacgtcaacagcggctgccagctgctgccccagcaccaggtgcccgcgtactgagtccgcgcgcggggcgcatgcgcggccaccctccccaaggggcgggctcgcgggggggtgtcgtgggcgccccggacttggagagggtgcggccctggggaccccccctccccgagtgtgcccaggaactcagagagggcgcggcccccggggattccccccccccgagggtgcccaggactcgacaagctggaccccctgctcccgggggggcgagcgcatgacccccccgccctcgcgctgcctctttcccccgcgcggccgccccgtgttgcacaaacccgcgcgtctcggctgcccctttgtacaccgcgccgcggaagggggctccgagggggcgcagcctcaaaccctgcctttcctttacttttacttttttttttttttctttggaagagagaagaacagagtgttcgattctgccctatttatgtttctactcgggaacaaacgttggttgtgtgtgtgtgtgttttcttgtgttggttttttaaagaaatgggaagaagaaaaaaaaattctccgcccctttcctcgatctcgctccccccttcggttctttcgaccggtcccccctcccttttttgttctgttttgttttgttttgctacgagtccacattcctgtttgtaatccttggttcgcccggttttctgttttcagtaaagtctcgttacgcca

SEQ ID NO: 2 - protein, 347 aa, Homo sapiens
METPFYGDEALSGLGGGASGSGGSFASPGRLFPGAPPTAAAGSMMKKDALTLSLSEQVAAALKPAAAPPPTPLRADGAPSAAPPDGLLASPDLGLLKLASPELERLIIQSNGLVTTTPTSSQFLYPKVAASEEQEFAEGFVKALEDLHKQNQLGAGAAAAAAAAAAGGPSGTATGSAPPGELAPAAAAPEAPVYANLSSYAGGAGGAGGAATVAFAAEPVPFPPPPPPGALGPPRLAALKDEPQTVPDVPSFGESPPLSPIDMDTQERIKAERKRLRNRIAASKCRKRKLERISRLEEKVKTLKSQNTELASTASLLREQVAQLKQKVLSHVNSGCQLLPQHQVPAY

SEQ ID NO: 3 - protein, 304 aa, Homo sapiens
MMKKDALTLSLSEQVAAALKPAAAPPPTPLRADGAPSAAPPDGLLASPDLGLLKLASPELERLIIQSNGLVTTTPTSSQFLYPKVAASEEQEFAEGFVKALEDLHKQNQLGAGAAAAAAAAAAGGPSGTATGSAPPGELAPAAAAPEAPVYANLSSYAGGAGGAGGAATVAFAAEPVPFPPPPPPGALGPPRLAALKDEPQTVPDVPSFGESPPLSPIDMDTQERIKAERKRLRNRIAASKCRKRKLERISRLEEKVKTLKSQNTELASTASLLREQVAQLKQKVLSHVNSGCQLLPQHQVPAY

SEQ ID NO: 4 - RNA, 25 nt, Homo sapiens
ucaguaaagucuucguuacgccaaa

SEQ ID NO: 5 - RNA, 27 nt, Homo sapiens
aaagucauuucagaagcaaugcgguuu

SEQ ID NO: 6 - RNA, 25 nt, Homo sapiens
uccuguuccguaauccuugguucgc

SEQ ID NO: 7 - RNA, 25 nt, Homo sapiens
cgcaguuccucuacccuaagguggc

SEQ ID NO: 8 - RNA, 25 nt, Homo sapiens
cgcaguuccucuacccuaagguggc

SEQ ID NO: 9 - RNA, 27 nt, Homo sapiens
augcgucaaggagaugggauuccaccg

SEQ ID NO: 10 - DNA, 28 bp, Homo sapiens
ctcgccattggctcgaggtgacgtcgca

SEQ ID NO: 11 - DNA, 28 bp, Homo sapiens
ctcgccattggctcgaggtgacgtcgcg

SEQ ID NO: 12 - DNA, 28 bp, Homo sapiens
cagaaactgcccggcaatccaagctggg

SEQ ID NO: 13 - DNA, 20 bp, Homo sapiens
catgacgtcaacccacaatg

SEQ ID NO: 14 - DNA, 20 bp, Homo sapiens
atagaagggcgtttccatcc

(19) United States
(12) Patent Application Publication
(10) Pub. No.: US 2010/0266579 A1
(43) Pub. Date: Oct. 21, 2010

(54) TREATMENT OF INFLAMMATORY DISEASES

(76) Inventors: Terence Cook, London (GB); Jacques Behmoaras, London (GB); Timothy Aitman, London (GB)

Correspondence Address: KLARQUIST SPARKMAN, LLP, 121 SW SALMON STREET, SUITE 1600, PORTLAND, OR, 97204, US

(21) Appl. No.: 12/738,920

(22) PCT Filed: Oct. 21, 2008
(86) PCT No.: PCT/GB08/03563

§ 371 Date: Jun. 22, 2010

(30) Foreign Application Priority Data
Oct. 26, 2007 (GB) 0720976.0

(51) Int. Cl. A61K 39/395; C07H 21/02; C07K 2/00; C07K 16/00; A61K 31/7105; A61K 38/02; C12Q 1/68; G06G 7/48; A61P 37/00; A61P 29/00; A61P 19/02; A61P 9/00; A61P 3/10; A61P 1/04; A61P 13/12; A61P 25/02

(52) U.S. Cl. 424/130.1; 536/24.5; 530/350; 530/387.1; 514/44 A; 514/1.1; 435/6; 703/11; 514/16.8; 514/16.4; 514/6.9; 514/13.2; 514/15.4; 514/18.2

(57) ABSTRACT

We describe the involvement of JunD in the activation of macrophages and the association of JunD in inflammatory diseases and conditions.

The invention relates to agents that inhibit the expression or activity of JunD; the use of said agents in the treatment of inflammatory diseases; and a diagnostic assay for the detection of JunD in a subject that is suffering from or has a genetic predisposition to an inflammatory disease.

There are many situations where the suppression of an immune response is desirable. For example autoimmune diseases and allergic responses are enhanced immune responses to specific antigens which result in pathological conditions (e.g. psoriasis, diabetes, asthma, rheumatoid arthritis, anaphylactic shock, and systemic lupus erythematosus) or discomfort (e.g. allergic rhinitis). These result in a considerable reduction in quality of life and in some situations the response can be life threatening. Therefore, in autoimmune and allergic diseases an aim is to suppress specific immune responses against the autoantigen or allergen.

Inflammation is a complex reaction of the body responding to damage of its cells and vascularised tissues. The inflammatory reaction is phylogenetically and ontogenetically the oldest defence mechanism and both the innate and adaptive immune systems in vertebrates are triggered to destroy the agent(s) that provoke inflammation. Inflammation can be acute or chronic. An acute inflammatory response is an immediate response by the immune system to a harmful agent. The response includes vascular dilatation, endothelial and neutrophil cell activation. An acute inflammatory response will either resolve or develop into chronic inflammation. Chronic inflammation is an inflammatory response of prolonged duration, weeks, months, or even indefinitely, whose extended time course is provoked by the persistence of the causative stimulus to inflammation within the tissue. The inflammatory process inevitably causes tissue damage. The exact nature, extent and time course of chronic inflammation is variable, and depends on a balance between the causative agent and the attempts of the body to remove it. Aetiological agents producing chronic inflammation include, but are not limited to: infectious organisms that can avoid or resist host defences and so persist in the tissue for a prolonged period; infectious organisms that are not innately resistant but persist in damaged regions where they are protected from host defences; irritant non-living foreign material that cannot be removed by enzymatic breakdown or phagocytosis; or where the stimuli is a “normal” tissue component, causing an auto-immune disease.

There is a vast array of diseases exhibiting a chronic inflammatory component. These include but are not limited to: inflammatory joint diseases (e.g., rheumatoid arthritis, osteoarthritis, polyarthritis and gout), chronic inflammatory connective tissue diseases (e.g., systemic lupus erythematosus, scleroderma, Sjorgen's syndrome, poly- and dermatomyositis, vasculitis, mixed connective tissue disease (MCTD), tendonitis, synovitis, bacterial endocarditis, osteomyelitis and psoriasis); chronic inflammatory lung diseases (e.g., chronic respiratory disease, pneumonia, fibrosing alveolitis, chronic bronchitis, chronic obstructive pulmonary disease (COPD), bronchiectasis, emphysema, silicosis and other pneumoconiosis and tuberculosis); chronic inflammatory bowel and gastro-intestinal tract inflammatory diseases (e.g., ulcerative colitis and Crohn's disease); chronic neural inflammatory diseases (e.g., chronic inflammatory demyelinating polyradiculoneuropathy, chronic inflammatory demyelinating polyneuropathy, multiple sclerosis, Guillan-Barre Syndrome and myasthemia gravis); other inflammatory diseases (e.g., mastitis, laminitis, laryngitis, chronic cholecystitis, Hashimoto's thyroiditis, inflammatory breast disease); chronic inflammation caused by an implanted foreign body in a wound; and including chronic inflammatory renal diseases including crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis. Diabetic nephropathy may also have a chronic inflammatory component and chronic inflammatory responses are involved in the rejection of transplanted organs.

The mediators of chronic inflammation are both cellular and humoral. Cellular responses include the infiltration of monocytes, macrophages and lymphocytes to the site of inflammation with concomitant tissue damage, angiogenesis and fibrosis. Humoral responses include the production antibodies, for example auto-antibodies and pro-inflammatory cytokines that maintain the inflammatory response. Monocytes and macrophages are phagocytes and their role is to engulf and destroy debris and invading pathogenic organisms and to promote the activity of lymphocytes to produce cytokines and antibodies to the inflammatory agent. The infiltration of monocytes into a site of inflammation results in differentiation to an activated macrophage. The activated macrophage can persist from many months to years in chronic inflammatory diseases.

This disclosure relates to the involvement of JunD in macrophage activation, in particular the over expression of JunD in pathological inflammatory conditions.

JunD is expressed as several isoforms differing in their content of the N-terminal peptide (Okazaki et al. (1998) Two proteins translated by alternative usage of initiation codons in mRNA encoding a JunD transcriptional regulator. Biochem Biophys Res Commun. 250 p 347-53; Short and Pfarr (2002) Translational Regulation of the JunD Messenger RNA. J. Biol. Chem., Vol. 277, p 32697-32705—both incorporated in their entirety herein by reference). It has been shown that Jun N-terminal kinase (JNK) phosphorylates both major isoforms, as the N-terminal JNK-docking domain is intact in both (Yazgan and Pfarr (2002) Regulation of two JunD isoforms by Jun N-terminal kinases J. Biol. Chem. 277 p 29710-29718).

Crescentic glomerulonephritis is an example of an inflammatory disease and is a major cause of kidney failure for which the underlying molecular basis is largely unknown. In previous studies, we mapped several major susceptibility genes, Crgn1-7, for crescentic glomerulonephritis in the Wistar Kyoto (WKY) rat. Our disclosure illustrates by combined congenic and microarray studies and by comparative functional genomics studies that the Ap1 transcription factor JunD contributes to glomerulonephritis susceptibility and is a major determinant of macrophage activity in humans.

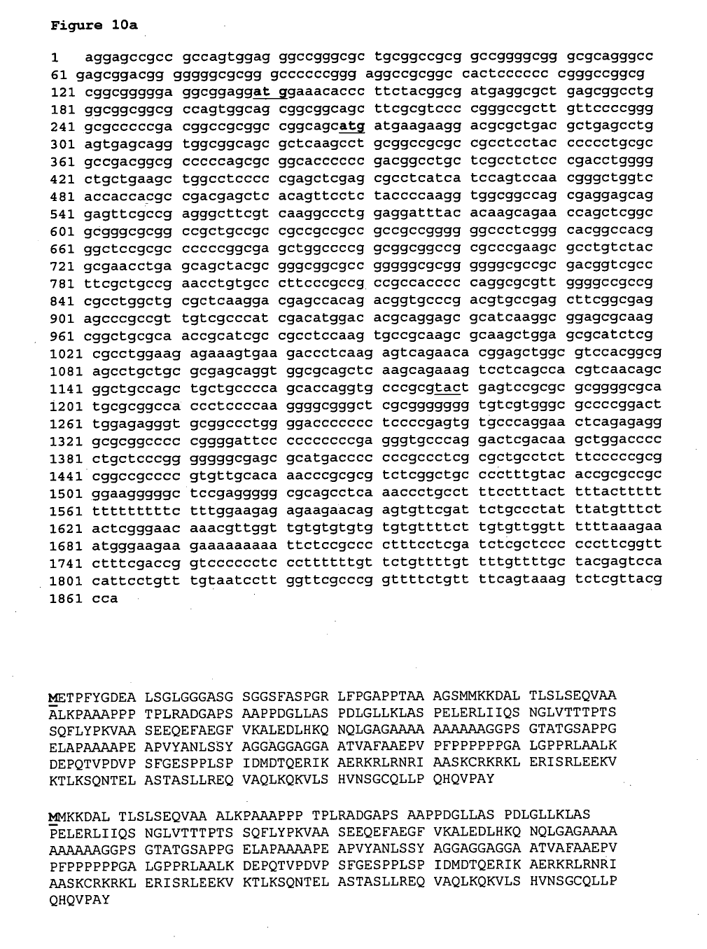

According to an aspect of the invention there is provided an agent that inhibits the expression or activity of a nucleic acid molecule or polypeptide encoded by said nucleic acid molecule wherein said nucleic acid molecule or polypeptide is selected from the group consisting of:
i) a nucleic acid molecule comprising the nucleic acid sequence in FIG. 10a;
ii) a nucleic acid molecule that hybridizes under stringent hybridization conditions to the nucleic acid sequence as represented in FIG. 10a and which encodes a polypeptide that has the activity associated with JunD;
iii) a polypeptide encoded by a nucleic acid molecule as defined in i) and ii) above;
iv) a polypeptide as represented by the amino acid sequence in FIG. 10b or 10c, wherein said agent is for use as a pharmaceutical.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, N.Y., 1993). The Tₘ is the temperature at which 50% of a given strand of a nucleic acid molecule is hybridized to its complementary strand. The following is an exemplary set of hybridization conditions and is not limiting:

Very High Stringency (Allows Sequences that Share at Least 90% Identity to Hybridize)
Hybridization: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridize)
Hybridization: 5×−6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridize)
Hybridization: 6×SSC at RT to 55° C. for 16-20 hours
Wash at least twice: 2×−3×SSC at RT to 55° C. for 20-30 minutes each.

In a preferred embodiment of the invention said agent is an inhibitory RNA; preferably a small inhibitory RNA (siRNA).

A number of techniques have been developed in recent years which claim to specifically ablate genes and/or gene products. For example, the use of anti-sense nucleic acid molecules to bind to and thereby block or inactivate target mRNA molecules is an effective means to inhibit gene expression. A technique to specifically ablate gene function is through the introduction of double stranded RNA, also referred to as small inhibitory or interfering RNA (siRNA), into a cell which results in the destruction of mRNA complementary to the sequence included in the siRNA molecule. The siRNA molecule comprises two complementary strands of RNA (a sense strand and an antisense strand) annealed to each other to form a double stranded RNA molecule. The siRNA molecule is typically derived from exons of the gene which is to be ablated. The mechanism of RNA interference is being elucidated. Many organisms respond to the presence of double stranded RNA by activating a cascade that leads to the formation of siRNA. The presence of double stranded RNA activates a protein complex comprising RNase III which processes the double stranded RNA into smaller fragments (siRNAs, approximately 21-29 nucleotides in length) which become part of a ribonucleoprotein complex. The siRNA acts as a guide for the RNase complex to cleave mRNA complementary to the antisense strand of the siRNA thereby resulting in destruction of the mRNA.

In a preferred embodiment of the invention inhibitory RNA molecule is at least 18 base pairs in length.

In a further preferred embodiment of the invention said inhibitory RNA molecule is between 19 bp and 1000 bp in length. More preferably the length of said inhibitory RNA molecule is at least 30 bp; at least 40 bp; at least 50 bp; at least 60 bp; at least 70 bp; at least 80 bp; or at least 90 bp.

In a yet further preferred embodiment of the invention said inhibitory RNA molecule is at least 100 bp; at least 200 bp; at least 300 bp; at least 400 bp; at least 500 bp; at least at least 600 bp; at least 700 bp; at least 800 bp; at least 900 bp; or at least 1000 bp in length.

Preferably said inhibitory RNA molecule is between 18 bp and 29 bp in length. More preferably still said inhibitory RNA molecule is between 21 by and 27 bp in length. Preferably said inhibitory RNA molecule is about 21 bp in length.

In a preferred embodiment of the invention wherein said siRNA is at least one selected from the group consisting of:

5′- Phos/UCAGUAAAGUCUUCGUUACGCCAAA -3′
3′- AAAGUCAUUUCAGAAGCAAUGCGGUUU -5′
5′- Phos/UCCUGUUCCGUAAUCCUUGGUUCGC -3′
3′- UAAGGACAAGGCAUUAGGAACCAAGCG
5′- Phos/CGCAGUUCCUCUACCCUAAGGUGGC -3′
3′- AUGCGUCAAGGAGAUGGGAUUCCACCG -5′

Preferably, said agent comprises at least 2 siRNAs selected from said group; preferably said agent comprises each of said siRNAs from the group.

In an alternative preferred embodiment of the invention said agent is an antisense nucleic acid molecule or oligonucleotide.

As used herein, the term “antisense oligonucleotide” or “antisense” describes an oligonucleotide that is an oligoribonucleotide, oligodeoxyribonucleotide, modified oligoribonucleotide, or modified oligodeoxyribonucleotide which hybridizes under physiological conditions to DNA comprising a particular gene or to an mRNA transcript of that gene and thereby, inhibits the transcription of that gene and/or the translation of that mRNA. The antisense molecules are designed so as to interfere with transcription or translation of a target gene upon hybridization with the target gene. Those skilled in the art will recognize that the exact length of the antisense oligonucleotide and its degree of complementarity with its target will depend upon the specific target selected, including the sequence of the target and the particular bases which comprise that sequence.

It is preferred that the antisense oligonucleotide be constructed and arranged so as to bind selectively with the target under physiological conditions, i.e., to hybridize substantially more to the target sequence than to any other sequence in the target cell under physiological conditions.

In order to be sufficiently selective and potent for inhibition, such antisense oligonucleotides should comprise at least 7 (Wagner et al., Nature Biotechnology 14:840-844, 1996) and more preferably, at least 15 consecutive bases which are complementary to the target. Most preferably, the antisense oligonucleotides comprise a complementary sequence of 20-30 bases.

Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. The 3′-untranslated regions are known to contain cis acting sequences which act as binding sites for proteins involved in stabilising mRNA molecules. These cis acting sites often form hair-loop structures which function to bind said stabilising proteins. A well known example of this form of stability regulation is shown by histone mRNA's, the abundance of which is controlled, at least partially, post-transcriptionally.

The term “antisense oligonucleotides” is to be construed as materials manufactured either in vitro using conventional oligonucleotide synthesising methods which are well known in the art or oligonucleotides synthesised recombinantly using expression vector constructs.

In a further preferred embodiment of the invention said inhibitory RNA or said antisense nucleic acid molecule is modified.

The term “modified” as used herein describes a nucleic acid molecule in which;
i) at least two of its nucleotides are covalently linked via a synthetic internucleoside linkage (i.e., a linkage other than a phosphodiester linkage between the 5′ end of one nucleotide and the 3′ end of another nucleotide). Alternatively or preferably said linkage may be the 5′ end of one nucleotide linked to the 5′ end of another nucleotide or the 3′ end of one nucleotide with the 3′ end of another nucleotide; and/or
ii) a chemical group, such as cholesterol, not normally associated with nucleic acids has been covalently attached to the double stranded nucleic acid.
iii) Preferred synthetic internucleoside linkages are phosphorothioates, alkylphosphonates, phosphorodithioates, phosphate esters, alkylphosphonothioates, phosphoramidates, carbamates, phosphate triesters, acetamidates, peptides, and carboxymethyl esters.

The term “modified” also encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2; azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N-6-methyladenine; 4-acetylcytosine, 5-(carboxyhydroxylmethyl) uracil; 5-fluorouracil; 5-bromouracil; 5-carboxymethylaminomethyl-2-thiouracil; 5 carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2 methylthio-N-6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-on/acetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Modified double stranded nucleic acids also can include base analogs such as C-5 propyne modified bases (see Wagner et al., Nature Biotechnology 14:840-844, 1996).

In a further alternative preferred embodiment of the invention said agent is a peptide; preferably a modified peptide antagonist.

In a preferred method of the invention said peptide is at least 6 amino acid residues in length. Preferably the length of said peptide is selected from the group consisting of: at least 7 amino acid residues; 8, 9, 10, 11, 12, 13, 14, 15, 16, 17, 18, 19, or 20 amino acid residues in length. Alternatively the length of said peptide is at least 20 amino acid residues; 30; 40; 50; 60; 70; 80; 90; or 100 amino acid residues in length.

It will be apparent to one skilled in the art that modification to the amino acid sequence of peptide agents could enhance the binding and/or stability of the peptide with respect to its target sequence. In addition, modification of the peptide may also increase the in vivo stability of the peptide thereby reducing the effective amount of peptide necessary to inhibit JunD. This would advantageously reduce undesirable side effects which may result in vivo. Modifications include, by example, acetylation and amidation. Alternatively or preferably, said modification includes the use of modified amino acids in the production of synthetic forms of peptides. It will be apparent to one skilled in the art that modified amino acids include, 4-hydroxyproline, 5-hydroxylysine, N⁶-acetyllysine, N⁶-methyllysine, N⁶,N⁶-dimethyllysine, N⁶,N⁶,N⁶-trimethyllysine, cyclohexyalanine, D-amino acids, ornithine. Other modifications include amino acids with a C₂, C₃ or C₄ alkyl R group optionally substituted by 1, 2 or 3 substituents selected from halo (e.g. F, Br, I), hydroxy or C₁-C₄ alkoxy. Modifications also include, by example, acetylation and amidation of amino and carboxy-terminal amino acids. It will also be apparent to one skilled in the art that peptides could be modified by cyclisation. Cyclisation is known in the art, (see Scott et al Chem Biol (2001), 8:801-815; Gellerman et al J. Peptide Res (2001), 57: 277-291; Dutta et al J. Peptide Res (2000), 8: 398-412; Ngoka and Gross J Amer Soc Mass Spec (1999), 10:360-363.

In a preferred embodiment of the invention said agent is an antibody or antibody fragment.

Various fragments of antibodies are known in the art, e.g. Fab, Fab₂, F(ab′)₂, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab₂ fragment comprises two joined Fab fragments. When these two fragments are joined by the immunoglobulin hinge region, a F(ab′)₂ fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No. 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof. A fragment of an antibody or immunoglobulin can also have bispecific function as described above. Methods to deliver proteins, peptides, antibodies and antibody fragments to cells intracellularly are known in the art; for example see WO2007/064727; WO2004/030610; WO03/095641; WO02/07671; WO01/43778; WO96/40248; and WO94/01131 each of which is incorporated by reference in their entirety.

According to a further aspect of the invention there is provided a composition comprising an agent according to the invention. Preferably said composition is a pharmaceutical composition.

In a preferred embodiment of the invention said composition further includes a carrier.

When administered the compositions of the present invention are administered in pharmaceutically acceptable preparations. Such preparations may routinely contain pharmaceutically acceptable concentrations of salt, buffering agents, preservatives, compatible carriers and supplementary anti-inflammatory agents.

The compositions of the invention can be administered by any conventional route, including injection or by gradual infusion over time. Treatment may be topical or systemic. The administration may, for example, be oral, intravenous, intraperitoneal, intramuscular, intracavity, subcutaneous, transdermal, transepithelial or intra bone marrow administration.

In a preferred embodiment of the invention treatment involves isolation of bone marrow-derived macrophages (BMDM) from the patient, treatment ex vivo to inhibit JunD activity and reintroduction to the patient at the site of inflammation.

In preferred embodiments of the invention said composition includes a second agent complexed or associated with the anti-inflammatory agent to facilitate the delivery of the agent to a cell. Preferably said agent is a liposome, immuno-liposome, dendrimer or polylysine-transferrine-conjugate.

The compositions of the invention are administered in effective amounts. An “effective amount” is that amount of a composition that alone, or together with further doses, produces the desired response. In the case of treating a particular disease, such as an inflammatory disease, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of an agent according to the invention for producing the desired response in a unit of weight or volume suitable for administration to a patient.

The doses of the agent according to the invention administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of nucleic acid agents of between 1 nM-1 μM generally will be formulated and administered according to standard procedures. Preferably doses can range from 1 nM-500 nM, 5 nM-200 nM, and 10 nM-100 nM. Other protocols for the administration of compositions will be known to one of ordinary skill in the art, in which the dose amount, schedule of injections, sites of injections, mode of administration and the like vary from the foregoing. The administration of compositions to mammals other than humans, (e.g. for testing purposes or veterinary therapeutic purposes), is carried out under substantially the same conditions as described above. A subject, as used herein, is a mammal, preferably a human, and including a non-human primate, cow, horse, pig, sheep, goat, dog, cat or rodent.

When administered, the pharmaceutical preparations of the invention are applied in pharmaceutically-acceptable amounts and in pharmaceutically-acceptable compositions. The term “pharmaceutically acceptable” means a non-toxic material that does not interfere with the effectiveness of the biological activity of the active ingredients. Such preparations may routinely contain salts, buffering agents, preservatives, compatible carriers, and optionally other therapeutic agents' (e.g. anti-inflammatory agents such as steroids, non-steroidal anti-inflammatory agents). When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

Compositions may be combined, if desired, with a pharmaceutically-acceptable carrier. The term “pharmaceutically-acceptable carrier” as used herein means one or more compatible solid or liquid fillers, diluents or encapsulating substances which are suitable for administration into a human. The term “carrier” in this context denotes an organic or inorganic ingredient, natural or synthetic, with which the active ingredient is combined to facilitate the application, (e.g. liposome or immuno-liposome). The components of the pharmaceutical compositions also are capable of being co-mingled with the molecules of the present invention, and with each other, in a manner such that there is no interaction which would substantially impair the desired pharmaceutical efficacy.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion or as a gel. Compositions may be administered as aerosols and inhaled.

Compositions suitable for parenteral administration conveniently comprise a sterile aqueous or non-aqueous preparation of agent, which is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

According to a further aspect of the invention there is provided a method to diagnose a subject suffering or having a predisposition to an inflammatory disease or condition comprising:
i) providing an isolated sample from a subject comprising a cell to be tested;
ii) determining the expression of a nucleic acid molecule or polypeptide encoded by a nucleic acid molecule comprising the nucleic acid sequence as represented in FIG. 10a or the amino acid sequence as represented in FIG. 10b or 10c;
iii) comparing the expression of said nucleic acid molecule or said polypeptide in said sample to a control sample; and
iv) determining the level of expression in said sample as an indicator of the existence or susceptibility of said subject to said inflammatory disease or condition.

In a preferred method of the invention said subject is human.

In a preferred method of the invention said disease is an auto-immune disease.

In a further preferred method of the invention said disease or condition is selected from the group consisting of: type 1 diabetes (e.g. diabetic nephropathy), rheumatoid arthritis, osteoarthritis, polyarthritis, gout, systemic lupus erythematosus, scleroderma, Sjorgen's syndrome, poly- and dermatomyositis, vasculitis, tendonitis, synovitis, bacterial endocarditis, osteomyelitis, psoriasis, pneumonia, fibrosing alveolitis, chronic bronchitis, chronic obstructive pulmonary disease (COPD), bronchiectasis, emphysema, silicosis, tuberculosis, ulcerative colitis and Crohn's disease, chronic inflammatory demyelinating polyradiculoneuropathy, chronic inflammatory demyelinating polyneuropathy, multiple sclerosis, Guillan-Barre Syndrome and myasthemia gravis, mastitis, laminitis, laryngitis, chronic cholecystitis, Hashimoto's thyroiditis, and including chronic inflammatory renal disease.

In a preferred method of the invention said disease is an inflammatory renal disease selected from the group consisting of glomerulonephritis, crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis and tubulointerstitial nephritis.

In a preferred method of the invention said method includes the identification of a treatment regime that would benefit said subject.

Assays to detect the expression of JunD mRNA and/or protein are well known in the art and include detection based on polymerase chain reaction; DNA sequencing to identify polymorphic sites (e.g. SNPs) in JunD, immunoassays to detect JunD for example an ELISA.

According to a further aspect of the invention there is provide a method to treat a subject suffering from or is predisposed to an inflammatory disease or condition comprising administering a pharmaceutically effective amount of an agent according to the invention.

In a preferred method of the invention said subject is human.

In a preferred method of the invention said disease is an auto-immune disease.

In a further preferred method of the invention said inflammatory disease or condition is selected from the group consisting of: type 1 diabetes, rheumatoid arthritis, osteoarthritis, polyarthritis, gout, systemic lupus erythematosus, scleroderma, Sjorgen's syndrome, poly- and dermatomyositis, vasculitis, tendonitis, synovitis, bacterial endocarditis, osteomyelitis, psoriasis, pneumonia, fibrosing alveolitis, chronic bronchitis, chronic obstructive pulmonary disease (COPD), bronchiectasis, emphysema, silicosis, tuberculosis, ulcerative colitis and Crohn's disease, chronic inflammatory demyelinating polyradiculoneuropathy, chronic inflammatory demyelinating polyneuropathy, multiple sclerosis, Guillan-Barre Syndrome and myasthemia gravis, mastitis, laminitis, laryngitis, chronic cholecystitis, Hashimoto's thyroiditis, and including chronic inflammatory renal disease.

In a preferred method of the invention said disease is an inflammatory renal disease selected from the group consisting of glomerulonephritis, crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis and tubulointerstitial nephritis.

According to a further aspect of the invention there is provided a method to prevent organ or tissue transplantation in a subject comprising administering an effective amount of an agent according to the invention to prevent or inhibit organ or tissue rejection.

Tissue engineering or transplantation has implications with respect to many areas of clinical and cosmetic surgery. More particularly, tissue engineering relates to the replacement and/or restoration and/or repair of damaged and/or diseased tissues to return the tissue and/or organ to a functional state. For example, and not by way of limitation, tissue engineering is useful in the provision of skin grafts to repair wounds occurring as a consequence of: contusions, or burns, or failure of tissue to heal due to venous or diabetic ulcers. Furthermore, tissue engineering is also practised during: replacement of joints through degenerative diseases such as arthritis; replacement of coronary arteries due to damage as a consequence of various environmental causes (e.g. smoking, diet) and/or congenital heart disease including replacement of arterial/heart valve; repair of gastric ulcers; replacement bone tissue resulting from diseases such as osteoporosis; replacement muscle and nerves as a consequence of neuromuscular disease or damage through injury. In addition, organ transplantation has for many years been an established surgical technique to replace damaged and/or diseased organs. The replacement of heart, lung, kidney, liver, bone marrow, and double organ transplantation of, for example, heart and lung, are relatively common procedures.

However, in both tissue engineering and organ transplantation a major obstacle to the successful establishment of a tissue graft or organ transplantation is the host's rejection of the donated tissue or organ as consequence the recipient's immune rejection of the foreign organ/tissue which is in part a chronic inflammatory response.

According to a further aspect of the invention there is provided an agent according to the invention for use in the treatment of an inflammatory disease or condition.

In a preferred embodiment of the invention said disease is an autoimmune disease.

In a preferred embodiment of the invention said inflammatory disease or condition is selected from the group consisting of: type 1 diabetes, rheumatoid arthritis, osteoarthritis, polyarthritis, gout, systemic lupus erythematosus, scleroderma, Sjorgen's syndrome, poly- and dermatomyositis, vasculitis, tendonitis, synovitis, bacterial endocarditis, osteomyelitis, psoriasis, pneumonia, fibrosing alveolitis, chronic bronchitis, chronic obstructive pulmonary disease (COPD), bronchiectasis, emphysema, silicosis, tuberculosis, ulcerative colitis and Crohn's disease, chronic inflammatory demyelinating polyradiculoneuropathy, chronic inflammatory demyelinating polyneuropathy, multiple sclerosis, Guillan-Barre Syndrome and myasthemia gravis, mastitis, laminitis, laryngitis, chronic cholecystitis, Hashimoto's thyroiditis, and including chronic inflammatory renal disease.

In a preferred of the invention said disease is an inflammatory renal disease selected from the group consisting of glomerulonephritis, crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis and tubulointerstitial nephritis.

According to a further aspect of the invention there is provided an agent according to the invention for use in the inhibition or prevention of organ/tissue rejection in transplantation therapy.

According to an aspect of the invention there is provided a screening method for the identification of an agent that has JunD inhibitory activity comprising the steps of:
i) providing a polypeptide encoded by a nucleic acid molecule selected from the group consisting of:
   a) a nucleic acid molecule comprising a nucleic acid sequence as represented in FIG. 10a;
   b) a nucleic acid molecule comprising nucleic acid sequences that hybridise to the sequence identified in (a) above under stringent hybridization conditions and which encodes a polypeptide that has JunD activity;
ii) providing at least one candidate agent to be tested;
iii) forming a preparation that is a combination of (i) and (ii) above; and
iv) testing the effect of said agent on the activity of JunD.

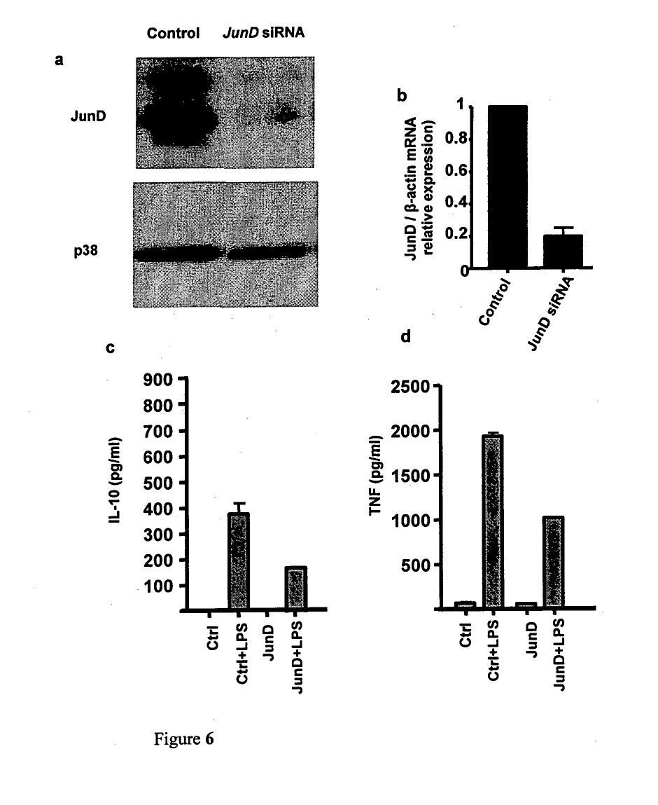

The skilled person is well aware of methods/assays to determine the activity of agents according to the invention. For example and not by way of limitation, DNA-binding activity of AP-1 (see www.activemotif.com/catalog/nuc_func/transam/ap1), or determining the downstream effects of JunD activation on pro-inflammatory cytokines such as IL-10 and TNF-α levels (see for example FIGS. 6c and 6d).

In a further preferred method of the invention said polypeptide is represented by the amino acid sequence in FIG. 10b or 10c.

In a preferred method of the invention said polypeptide is expressed by a cell wherein said cell is transformed or transfected with a nucleic acid molecule that encodes a Jun D polypeptide. Preferably said nucleic acid molecule is part of a vector adapted for recombinant expression of said nucleic acid molecule. Preferably said vector is provided with a promoter which enables the expression of said nucleic acid molecule to be regulated.

In a preferred method of the invention said cell is derived from a monocyte, preferably said cell is a macrophage; preferably an activated macrophage.

In a preferred method of the invention said agent is selected from the group consisting of: a siRNA, an antisense nucleic acid or oligonucleotide, a peptide.

According to a further aspect of the invention there is provided a modelling method to determine the association of an agent with a JunD polypeptide comprising the steps of:
i) providing computational means to perform a fitting operation between an agent and a polypeptide defined by the amino acid sequence in FIG. 10b or 10c; and
ii) analysing the results of said fitting operation to quantify the association between the agent and the JunD polypeptide.

The rational design of binding entities for proteins is known in the art and there are a large number of computer programs that can be utilised in the modelling of 3-dimensional protein structures to determine the binding of chemical entities to functional regions of proteins and also to determine the effects of mutation on protein structure. This may be applied to binding entities and also to the binding sites for such entities. The computational design of proteins and/or protein ligands demands various computational analyses which are necessary to determine whether a molecule is sufficiently similar to the target protein or polypeptide. Such analyses may be carried out in current software applications, such as the Molecular Similarity application of QUANTA (Molecular Simulations Inc., Waltham, Mass.) version 3.3, and as described in the accompanying User's Guide, Volume 3 pages. 134-135. The Molecular Similarity application permits comparisons between different structures, different conformations of the same structure, and different parts of the same structure. Each structure is identified by a name. One structure is identified as the target (i.e., the fixed structure); all remaining structures are working structures (i.e. moving structures). When a rigid fitting method is used, the working structure is translated and rotated to obtain an optimum fit with the target structure.

The person skilled in the art may use one of several methods to screen chemical entities or fragments for their ability to associate with a target. The screening process may begin by visual inspection of the target on the computer screen, generated from a machine-readable storage medium. Selected fragments or chemical entities may then be positioned in a variety of orientations, or docked, within the binding pocket.

Useful programs to aid the person skilled in the art in connecting the individual chemical entities or fragments include: CAVEAT (P. A. Bartlett et al, “CAVEAT: A Program to Facilitate the Structure-Derived Design of Biologically Active Molecules”. In Molecular Recognition in Chemical and Biological Problems”, Special Pub., Royal Chem. Soc., 78, pp. 182-196 (1989)). CAVEAT is available from the University of California, Berkeley, Calif. 3D Database systems such as MACCS-3D (MDL Information Systems, San Leandro, Calif.). This is reviewed in Y. C. Martin, “3D Database Searching in Drug Design”, J. Med. Chem., 35, pp. 2145-2154 (1992); and HOOK (available from Molecular Simulations, Burlington, Mass.). These citations are incorporated by reference.

Once the agent has been optimally selected or designed, as described above, substitutions may then be made in some of its atoms or side groups in order to improve or modify its binding properties. Generally, initial substitutions are conservative, i.e., the replacement group will have approximately the same size, shape, hydrophobicity and charge as the original group. The computational analysis and design of molecules, as well as software and computer systems are described in U.S. Pat. No. 5,978,740 which is included herein by reference.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

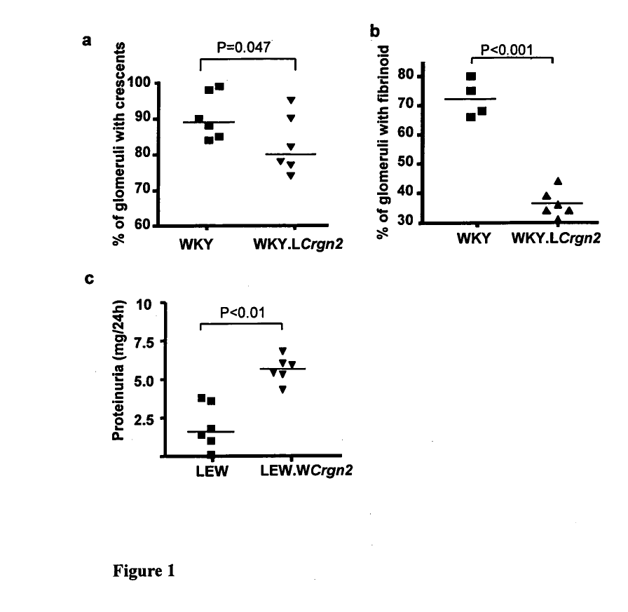

FIG. 1 illustrates quantitative measurements of glomerular crescents (a) and fibrinoid (b) as well as proteinuria (c) in parental (WKY and Lewis) and chromosome 16 congenic strains (WKY.LCrgn2, LEW.WCrgn2). Rats were sacrificed 10 d following the NTN induction and phenotypes were measured using at least 6 rats per strain.

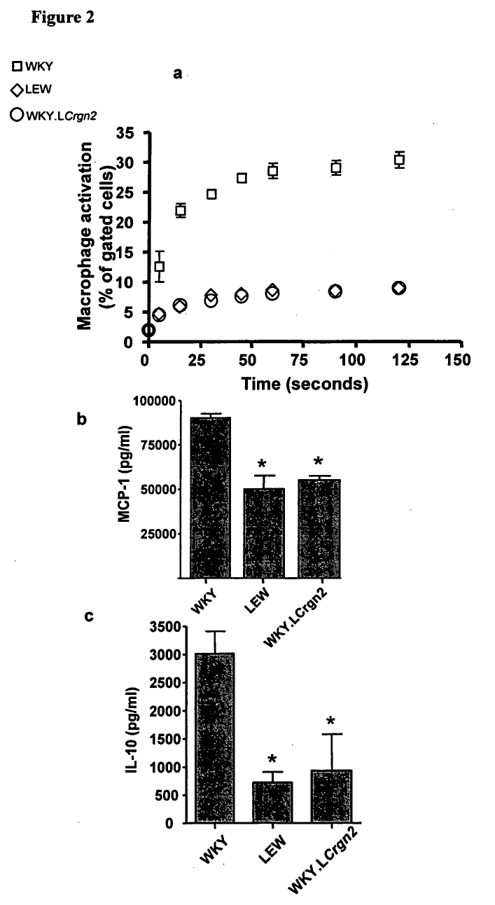

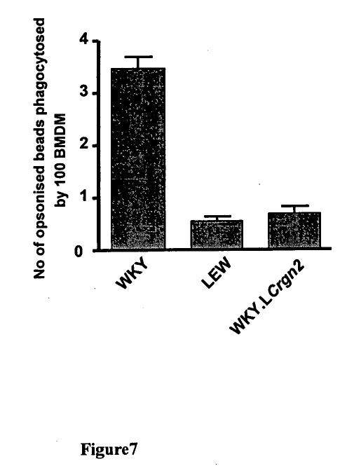

FIG. 2 illustrates macrophage activation and cytokine production of the parental (WKY and Lewis) and chromosome 16 congenic rats. Macrophage activation assessed by Fc receptor mediated phagocytosis and oxidization (a). 104 fluorescence events at 0, 5, 15 30, 45, 60, 90 and 120 seconds were measured after stimulating WKY, LEW and WKY.LCrgn2 BMDM (106 cells in triplicate, n=3 rats/strain) with Fc OxyBURST (240 μg/ml) in a FACSCalibur. ANOVA followed by Bonferroni test was applied and LEW and WKY. LCrgn2 BMDM showed significantly reduced activation compared to WKY at all time points. Macrophage activation was also assessed by bead phagocytosis (FIG. 7) and showed similar results. MCP-1 in control (unstimulated) (b) and IL-10 production in LPS (100 ng/ml) stimulated (c) WKY, LEW and WKY.LCrgn2 BMDM were measured by sandwich ELISA. *, p<0.001 compared to WKY; error bars represent s.e.m.

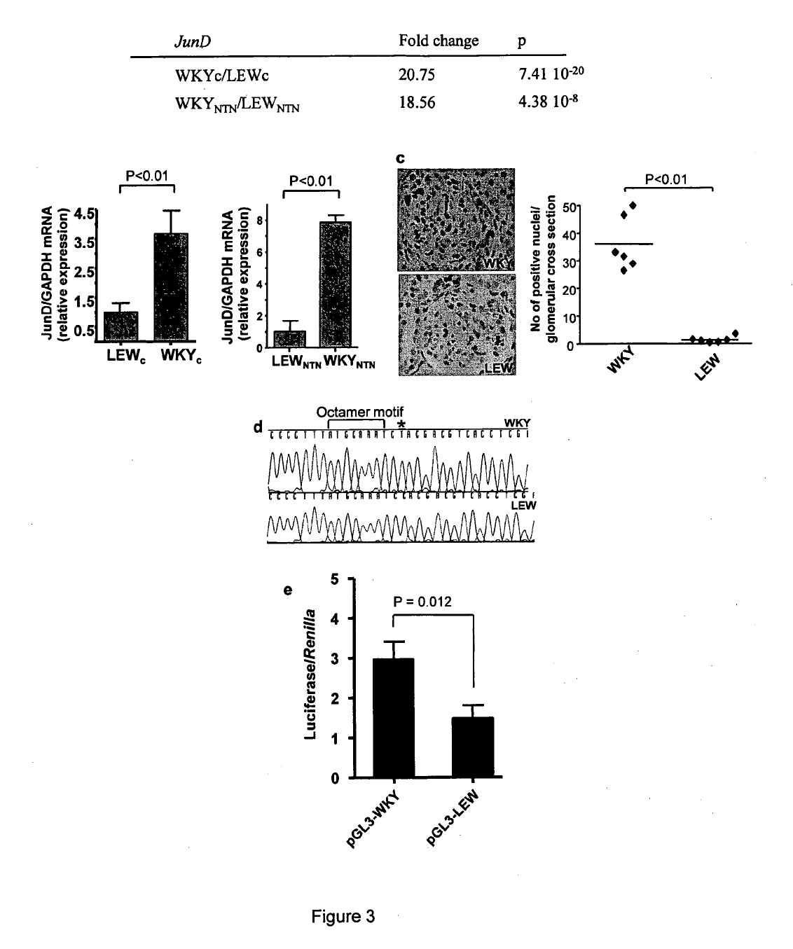

FIG. 3 illustrates combined gene expression and genetic mapping analysis identifies JunD as a candidate gene for NTN suscebtibility in rat. Microarray (a) and qRT-PCR (b) analysis showing that JunD is differentially expressed between WKY and Lewis control and NTN induced glomeruli (WKYc, LEWc, WKYNTN, and LEWNTN respectively). At least n=3 rats per strain and per condition (control or NTN) were used. Error bars represent s.e.m. Immunostaining for JunD protein in WKY and Lewis glomeruli following NTN induction (day 10) showed increased levels in the WKY rat (c). Sequence analysis of rat JunD promoter showed a C/T polymorphism (−210) in the vicinity of an octamer binding motif (−212); denoted with an asterisk (d). Luciferase assay was performed after transfecting Cos7 cells with pGL3-basic vector containing either the WKY or Lewis JunD promoters (pGL3-WKY and pGL3-LEW respectively, 300 bp upstream the TIS) (e). All constructs were co-tranfected with Renilla reniformis luciferase driven under SV40 promoter (pRL-SV40) to normalize for transfection efficiency; minimum and maximum promoter activities were determined by co-transfecting either PGL3-control or PGL3-basic with pRL-SV40 respectively. Firefly luciferase activity, normalized to Renilla luciferase was expressed relative to the activity of pGL3-basic vector. Error bars represent s.e.m for 5 different transfection experiments performed in replicates of 6.

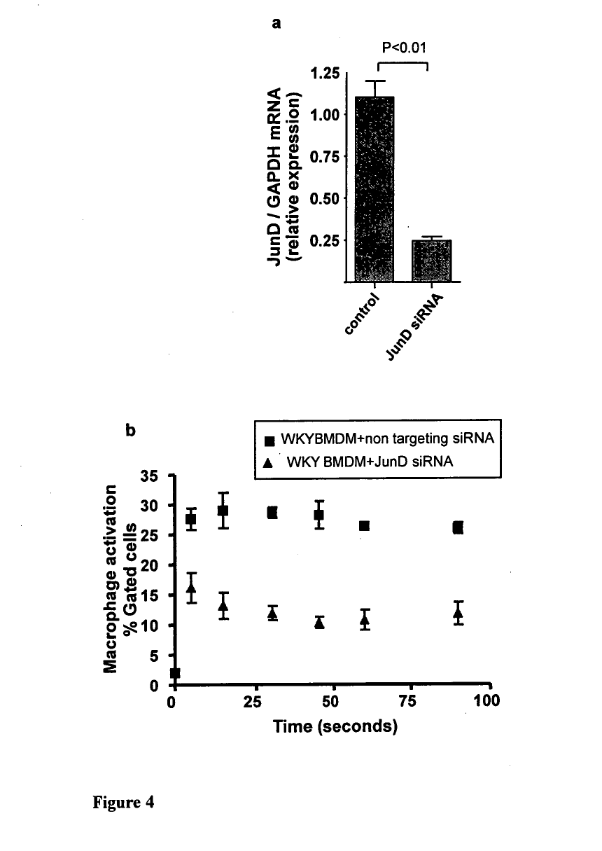

FIG. 4 illustrates JunD expression levels regulate Fc receptor mediated macrophage activity. WKY BMDM were cultured (106 cells in triplicate, n=3 rats) and transfected either with JunD siRNA or control (non-targeting) siRNA. 48 hours following transfection, JunD mRNA levels were measured by qRT-PCR (a) and macrophage activation was assessed by Fc Oxyburst assay (b). WKY BMDM with JunD knockdown showed significantly reduced activation compared to BMDM transfected with control siRNA at all time points. Error bars represent s.e.m.

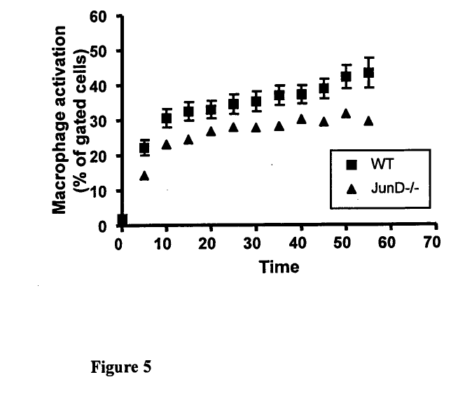

FIG. 5 illustrates NTN and related phenotypes in JunD−/− mice. Mice were immunized intraperitoneally with 0.2 mg of sheep IgG in a 50:50 mix with complete Freund's adjuvant. 5 mg of nephrotoxic globulin (NTS) was injected intravenously to both WT and JunD−/− mice 5 days after immunization (n=10/group). Mice were sacrificed at day 10 following NTS injection and glomerular crescents and thrombosis as well as serum creatinine, albumin and albuminuria were assessed. For the macrophage activation measurements, BMDM (106 cells in triplicate, n=5 mice/group) were cultured from WT and JunD−/− mice and FcOxyburst assay was performed subsequently. JunD−/− BMDM showed significantly reduced activation compared to WT at all time points.

FIG. 6 illustrates JunD expression levels regulate cell activity in human primary macrophages. Human macrophages were derived from elutriated monocytes by culturing the cells with M-CSF at 100 ng/ml for 3 days. Cells were then incubated either with JunD siRNA (100 nM) or control (non-targeting) siRNA (100 nM) for 48 h and incubated with LPS (10 ng/ml) for 24 h. Cells lysates were then subjected to JunD and p38 Western blotting and RNA extraction for human JunD qRT-PCR. IL-10 (c) and TNF-α (d) production were assessed by sandwich ELISA in cell supernatants.

FIG. 7 illustrates antibody-opsonised bead phagocytosis of the parental (WKY and LEW) and WKY.LCrgn2 rats. BMDM used in the Fc-oyburst assay (FIG. 2) were subjected to bead phagocytosis.

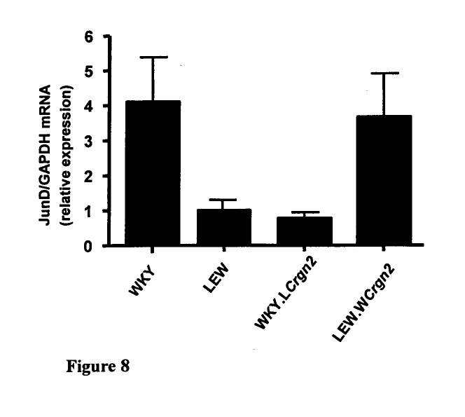

FIG. 8 illustrates JunD expression co-segragetes with the Crgn2 congenic interval. JunD expression was assessed by qRT-PCR in WKY, LEW, WKY.LCrgn2 and LEW, WCrgn2 BMDM. 3 rats per strain were used and error bars represent s.e.m

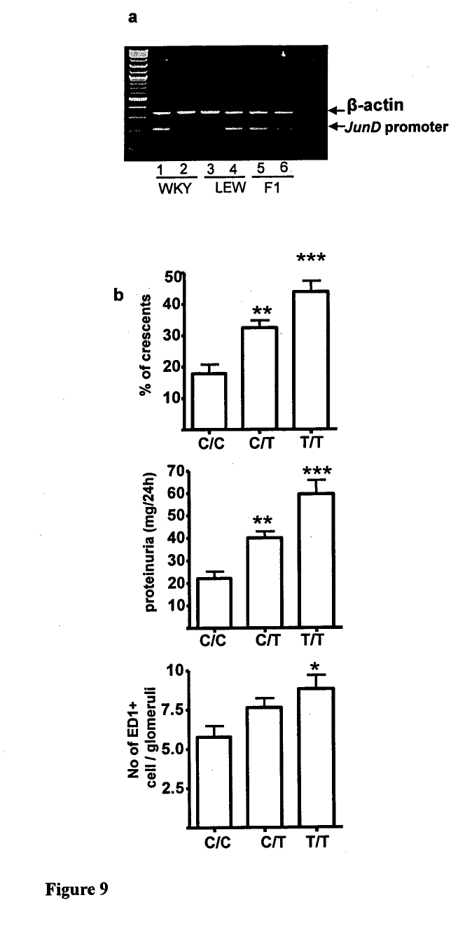

FIG. 9 illustrates rat JunD promoter polymorphism between WKY and LEW was used to genotype 177 F2 rats derived from WKY and LEW by PCR-based ARMS assay (a). PCR is illustrated here for WKY DNA (Lane 1, 2) LEW DNA (Lane 3, 4) and F1 DNA (Lane 5, 6) with WKY specific allele (T/T, lane 1, 3, 5); LEW specific allele (C/C, lane 2, 4, 6). Different NTN-related phenotypes were analyzed in 177 F2 rats according to their JunD polymorphism (b). All genotypes were compared to rats having the C/C genotype. *, p<0.05; **, p<0.01; ***, p<0.001. Error bars represent s.e.m

FIG. 10a is the nucleic acid sequence of human JunD; FIG. 10b is the amino acid sequence of full length human JunD; FIG. 10c is the amino acid sequence of a truncated JunD isoform;

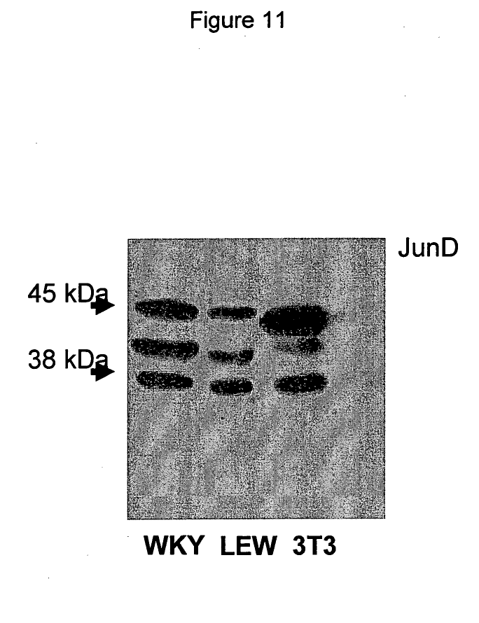

FIG. 11 the isoforms of JunD are illustrated using a western blot of mesangial nuclear extracts; and

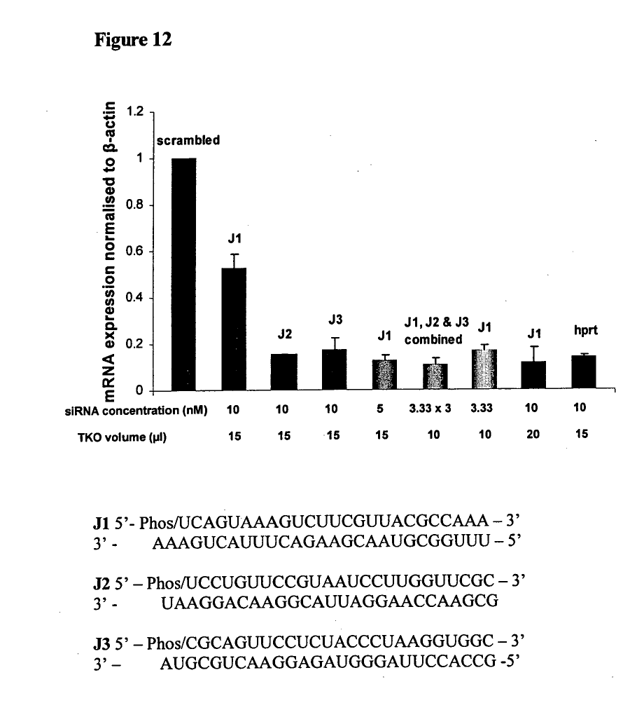

FIG. 12 illustrates in vitro knockdown of JunD in bone marrow derived macrophages (BMDM) using siRNA.

### Materials and Methods

### Animals

Wistar-Kyoto (WKY/NCrl) and Lewis (LEW/Crl) rats were purchased from Charles River (Margate, UK). Lewis rats were purchased from Harlan for microarray experiments. F1 rats were generated by intercrossing the two strains. JunD−/− mice used in this study have been previously described¹⁵. All mice were housed under pathogen-free conditions and all procedures were performed in accordance with the United Kingdom Animals (Scientific Procedures) Act.

### Generation of Congenic and Double Congenic Rat Lines

To prove the involvement of the chromosome 16 QTL in the NTN phenotype, 2 congenic rat lines were produced by introgression of segment of interest on chromosome 13, Crgn1 (D13Arb10-D13Rat 51) and on chromosome 16, Crgn2 (D16Rat88-D16Rat40) from the WKY donor onto the Lewis recipient genome and vice versa. Crgn1 and Crgn2 congenics on both WKY (WKY.LCrgn1, WKY.LCrgn2 respectively) and Lewis (LEW.WCrgn1, LEW.WCrgn2) genetic backgrounds were generated by backcrossing the F1 rats to WKY and Lewis parental strains for 9 generations, heterozygous rats for chromosome 16 and 13 linkage regions were crossed together to obtain the congenic lines. The genetic background of the congenic strains was tested by using 2 microsatellite markers outside the congenic region and 20 cM distant from each other in 20 autosomal chromosomes and the X chromosome.

We constructed a double congenic, i.e., a single strain in which both the LEW Crgn1 and Crgn2 were on the WKY genetic background and vice versa. The double congenic will be called the Chr 13/16 double congenic; it was constructed as follows. The Chr 16 and 13 congenic strains were crossed and this F1 was back-crossed to either Chr 16 or Chr 13 congenics. Rats that were homozygous for chromosome 13 and heterozygous for chromosome 16.

### NTN Induction

Nephrotoxic serum (NTS) was prepared in rabbits by standard methods. Nephrotoxic nephritis was induced in male rats by intravenous injection of 0.1 ml of NTS. Nine days later urine was collected by placing rats into metabolic cages for 24 hours with free access to food and water. Proteinuria was determined by the sulphosalicylic acid method³. On day 10 after induction of NTN, rats were killed under isoflurane anaesthesia and blood was collected from the abdominal aorta. Samples of kidney, skin, liver, colon and lung were fixed in 10% formal saline, processed and embedded in paraffin wax.

### Bone Marrow Derived Macrophage (BMDM) Culture, Fc Oxyburst and Bead Phagocytosis Assays

Femurs from adult rats and mice were isolated and flushed with Hanks buffer (Gibco).

Total bone marrow derived cells were plated cultured for 7 days in DMEM (Gibco) containing 25 mM Hepes (Sigma, UK), 25% L929 conditioned media, 25% decomplemented foetal bovine serum (F-539, M. B. Meldrum, Bourne End, UK), penicillin (100 U/ml, Invitrogen), streptomycin (100 μg/ml, Invitrogen), L-glutamine (2 mM, Invitrogen). These cells were characterized as macrophages by ED-1 staining. For Fc oxyburst assay, BMDM were counted in a hemocytometer. 10⁶ cells (in triplicate) were suspended in Krebs' Ringer's PBS (KRP buffer) with 1.0 mM Ca²⁺, 1.5 mM Mg²⁺ and 5.5 mM glucose, warmed to 37° C. and stimulated with Fc oxyBURST reagent (240 μg/ml, Invitrogen). Individual data points consisting of 10000 fluorescence events were collected at 0, 15, 30, 45, 60, 90 and 120 seconds in a FACSCalibur after a baseline fluorescence reading was taken to determine the intrinsic fluorescence of the unstimulated cells. % of fluorescent BMDM corresponds to % of activated gated cells following Fc-receptor mediated phagocytosis.

Macrophage bead phagocytosis was assessed as described³². Briefly, after differentiation, BMDM were harvested and cultured for two days in 8 well glass chamber slides (Nunc) at 10⁵ macrophages per well. Two hours prior to the addition of the beads, the cells were then washed in warm Hanks (Gibco) and serum free DMEM was added. Anti BSA-rabbit derived IgG (Sigma) opsonised and unopsonised 6 micron polystyrene beads (Polysciences) were then added to wells at 20 beads per target cell. Each condition was done in duplicate and the experiment was repeated on three separate rats. The chamber slides were then incubated at 37° C., 5% CO₂ for ten minutes washed in PBS and fixed in cold methanol for two minutes before a diffquik stain was performed. One hundred BMDM with or without ingested beads were then counted in a blinded manner and the number of beads ingested was noted.

Gene Expression Profiling and Quantitative RT-PCR (qRT-PCR)

Total RNA was extracted from WKY and LEW glomeruli and cRNA synthesized from 10 μg total RNA and purified as described. The Affymetrix Rat Genome U34 Set was used on RNA extracted from WKY and LEW glomeruli (n=3 rats per condition) with or without NTN induction as previously described³³. Rat BMDM and glomeruli RNA were extracted using Trizol (Invitrogen, UK) and JunD qRT-PCR was performed using gene specific primers as previously described³.

### ARMS (Amplification Refractory Mutation System) Analysis

For the UT polymorphism in the JunD promoter, PCR was performed using a WKY T allele specific reverse primer, 5′-CTCGCCATTGGCTCGAGGTGACGTCGCA-3′; or a LEW C allele specific primer 5′-CTCGCCATTGGCTCGAGGTGACGTCGCG-3′ with a common forward primer, 5′-CAGAAACTGCCCGGCAATCCAAGCTGGG-3′ together with B-actin primers. 2 PCR reactions were performed for a single DNA product (125 ng) using either WKY T or LEW C specific primers including B-actin primers as a control of PCR. Following amplification, PCR products were analysed on a 2.5% agarose gel and genotypes were determined according to the presence or absence of allele-specific bands.

### JunD Promoter Polymorphism and Luciferase Assay

JunD promoter polymorphism was detected by direct sequencing 100 ng of WKY and LEW genomic DNA using 5′-CATGACGTCAACCCACAATG-3′,5′-ATAGAAGGGCGTTTCCATCC-3′ forward and reverse primers respectively in a ABI3730xl (Applied Biosystems). At 48 hours before transfection, cos7 cells were seeded onto 96-well plates at a density of 2.5×10⁴ cells per well. In order to detect maximum and minimum promoter activities, cos7 cells were co-transfected either with pGL3-Control (200 ng, Promega) or pGL3-Basic (200 ng, Promega) together with 200 ng of internal control reporter Renilla reniformis luciferase driven under SV40 promoter (pRL-SV40; Promega) for normalizing for the transfection efficiency. Transfections were performed using Lipofectamine 2000 reagent (Invitrogen). In order to compare WKY and Lewis JunD promoter activities, 300 bp of the JunD promoter containing the C/T polymorphism was amplified by PCR from WKY and LEW genomic DNA where NheI and HindIII restriction sites were introduced in 5′ end of forward and reverse primers. The PCR products obtained from WKY and Lewis genomic DNA were subsequently cloned into pGL3-Basic vectors (named pGL3-WKY and pGL3-Lew respectively) were co-transfected into cos7 cells with 200 ng of pRL-SV40. After being washed in PBS, cells were resuspended in 250 μl of lysis buffer. Luciferase assay was performed using the dual luciferase assay system kit essentially according to the manufacturer's protocols (Promega). Values for the relative promoter activity were calculated from the ratio of firefly/Renilla luciferase activities using a luminometer (FluoStar,).

siRNA Inhibition of JunD Expression

Human primary macrophages were derived from elutriated monocytes by culturing the cells with M-CSF at 100 ng/ml (Wyeth, Boston, Mass.) in 10% heat-inactivated FCS RPMI 1640 for 3 days as previously described³⁴. WKY BMDM and human primary macrophages were plated in 6 well plates (10⁶ cells/well) in presence of DMEM 1× (Gibco,) for 24 hours and transfescted for 48 h with JunD siRNA (100 nM, siGENOME SMARTpool, Dharmacon, UK) and double stranded non targeting siRNA (100 nm, Ambion) using Dharmafect 1 (1/50, Dharmacon, UK) as a transfection reagent in OPTIMEM medium.

Bone marrow derived macrophages were extracted and pooled from 2-3 WKY rats and cultured in L929 conditioned full culture media. 1×10⁶ cells per well were transfected with combinations of JunD siRNA, scrambled SiRNA and as a positive control Hprt SiRNA. SiRNA was diluted in Optimum media and the same for the transfection reagent Trans IT TKO (Mirus) and incubated for 10-20 minutes at room temperature prior to transfecting cells. Each condition was done in triplicate. Cells were treated with trizol 24 hours later and expression of JunD and hprt measured against the housekeeping gene B-Actin by RT-PCR. The results are illustrated in FIG. 12.

JunD−/− Mice and Nephrotoxic Nephritis

The JunD−/− mice has a mixed C57BI6/129Sv background with exchange of the JunD sequence for lacZ. Genotypes were determined by PCR using primers for JunD and lacZ. NTN induction was performed in wild type (WT) and JunD−/− mice as previously described³⁵.

### Western Blot Analysis of JunD

Human macrophage cell extracts were resolved by SDS-PAGE and transferred to polyvinylidene difluoride membranes (Millipore, Bedford, Mass.), which were blocked for 1 h with blocking buffer (5% (w/v) fat-free milk and 0.1% (v/v) Tween 20 in PBS), followed by 1 h incubation with the JunD Ab (Santa Cruz, Calif.) diluted 1/1000 in blocking buffer. HRP-conjugated anti-rabbit IgG (Amersham Pharmacia Biotech) were used as a secondary Ab at a dilution of 1/2000. Bound Ab was detected using the ECL kit (Amersham Pharmacia Biotech) and was visualized using Hyperfilm MP (Amersham Pharmacia Biotech).

### Cytokine Determination by ELISA

Human IL-10, TNF-α (PharMingen International, Oxford, UK) and rat MCP-1, IL-10 (BD Biosciences, UK) sandwich ELISAs were carried out in macrophages plated in 6-well plates at a density of 10⁶ cells/well in accordance with the manufacturer's specifications.

### EXAMPLES

We first evaluated the effect of Crgn2 on NTN-related phenotypes in parental (WKY and LEW) and congenic lines on a WKY genetic background with introgression of LEW Crgn2 (WKY.LCrgn2) and vice versa (LEW.WCrgn2). Introgression of LEW Crgn2 on a WKY background significantly reduced glomerular crescent and fibrin whereas the reciprocal congenic animals (LEW.WCrgn2) showed increased proteinura suggesting that Crgn2 linkage region affects NTN susceptibility in the WKY rat (FIGS. 1a,b,c). LEW.WCrgn2 and WKY.LCrgn2 animals did not show significantly increased glomerular crescents and reduced proteinuria respectively (Data not shown). Crgn2 is also involved in macrophage activation and cytokine production as WKY.LCrgn2 bone-marrow derived macrophages (BMDM) rats showed reduced Fc receptor mediated macrophage activation (FIG. 2a FIG. 7) and Lipopolysaccharide (LPS) induced interleukin-10 (IL-10) and basal Monocyte chemoattractant protein-1 (MCP-1) production (FIG. 2b,c).

Global gene expression profiling was previously used combined with linkage analysis in order to positionnaly clone complex trait susceptibility genes in rat^{9,10}. Here, we carried out microarray analysis on WKY and LEW NTN induced (WKY_{NTN} and LEW_{NTN}) and normal (WKY_{c} LEW_{c}) glomeruli and identified JunD overexpression in glomeruli and BMDM (FIG. 3a, b and FIG. 8). WKY glomerular JunD overexpression is also characterised by increased JunD protein levels at day 10 following NTN induction (FIG. 3c). Sequence analysis of the WKY and LEW JunD promoters revealed a C/T polymorphism in the vicinity of an octamer motif in the rat JunD promoter, −210 by upstream the transcription initiation site (TIS) partly responsible of the inter-strain differential expression (FIG. 3d,e). JunD promoter UT polymorphism was used to genotype 177 F2 rats derived from WKY and LEW in a PCR-based ARMS assay to map JunD at the Crgn2 peak of linkage, co-segragating with the microsattelite marker D16Rat78. (FIG. 9). Moreover, JunD expression was found to be co-segregating with the Crgn2 congenic interval in both BMDM (FIG. 8) and glomeruli (data not shown). Based on these results, we hypothesised that over expression of JunD located at the Crgn2 peak of linkage in the NTN-susceptible WKY rat makes JunD the most likely candidate gene in the pathophysiology of experimentally induced glomerulonephritis in the WKY rat.

The activator protein-1 (AP-1) transcription factor JunD is ubiquitously expressed and reported to function as a negative regulator of cell proliferation, protective protein against apoptosis and oxidative stress¹¹⁻¹³. In addition, although JunD regulates the transcriptional activity of Th2 cytokines¹⁴, its role in inflammation mediated diseases and macrophage activation was not investigated. To ask whether JunD is involved in rat macrophage activation its expression levels were inhibited in WKY BMDM by siRNA and the subsequent macrophage activation was measured. Knockdown of JunD in WKY BMDM resulted in significantly reduced macrophage activation (FIG. 4) suggesting that JunD is specifically regulating macrophage activation in the Crgn2 linkage region.

JunD deficient mice (JunD−/−) are phenotypically normal despite growth defects¹⁵ JunD−/− BMDM showed reduced macrophage activation compared to wild type (WT) controls. NTN induction in the JunD−/− mice and WT controls showed a significant protection in the JunD−/− mice.

In response to cytokines and microbial products, macrophages express polarized functional properties including differential cytokine expression and Fc-receptor mediated activation. Classically activated macrophages (M1) promote type I immune responses in the initiation of the inflammation process, while alternatively activated (M2) promote type II immunity and are hyporesponsive to proinflammatory stimuli playing a role in tissue repair¹⁶⁻¹⁹. Previous studies highlighted important roles for key proteins regulating macrophage phenotypes²⁰. To ask whether JunD have a more general role in macrophage phenotype, we examined the cytokine secretion in LPS-treated human primary macrophages when JunD is knocked down. We observed that siRNA inhibition of JunD (FIG. 6a,b) in human primary macrophages prevented LPS-induced secretion of IL-10 and tumour necrosis factor-α (TNF-α) (FIG. 6c,d) showing that JunD mRNA levels directly regulate IL-10 and TNF-α secretion in human macrophages. JunD knock down did not change the interleukin-6 (IL-6) secretion (data not shown) suggesting that intracellular JunD mRNA levels regulate specific cytokine expression rather than a generalized transactivation.

Taken together, our observations suggest that JunD overexpression mapping to Crgn2 linkage region play an important role in the pathophysiology of Crgn by regulating macrophage activity. The NTN-susceptible WKY rats over express JunD in their BMDM and also show increased macrophage activation and LPS-induced IL-10 secretion. Furthermore, JunD knockdown in LPS-treated human primary macrophages led to reduced IL-10 secretion suggesting that JunD mRNA levels directly regulate IL-10 production in Crgn2. Although known as an immunosuppressive cytokine with anti-inflammatory effects, IL-10 processes immunostimulatory effects and was proposed as the basis of several antibody-mediated autoimmune disorders including systemic lupus erythematosus (SLE) which can cause kidney inflammation^{21,22}. High IL-10 production has been observed in macrophages from SLE patients in vitro showing also increased serum IL-10 levels²³⁻²⁵. This implies that macrophage JunD mRNA levels may play a critical role in the pathophysiology of SLE in humans and Crgn in the WKY rat by regulation of IL-10 expression and secretion.

Inflammatory roles of AP-1 proteins in macrophage activation have not been yet clarified. The amplitude and qualitative nature of macrophage activation are tightly regulated by a crosstalk among Jak-STAT, Toll-like receptor and ITAM dependant pathways²⁶. Although we propose a key regulatory role for JunD in macrophage activation in the pathophysiology of Crgn, the intracellular signalling pathways leading to overactivated JunD/AP-1 requires further investigation. The c-Jun amino-terminal kinase (JNK) pathway is activated by a wide variety of immunological stimuli including pro-inflammatory cytokines leading to phosphorylated-JNK activation of JunD^{27,28}. Accordingly, JNK mediated AP-1 activation and its pathogenic role in experimental anti-glomerular basement membrane disease in rats²⁹⁻³¹ highlights a potential therapeutic role of the inhibition of JunD/AP-1 in Crgn.

### REFERENCES

1. Tam, F. W. et al. Development of scarring and renal failure in a rat model of crescentic glomerulonephritis. Nephrology Dialysis Transplantation 14, 1658-66 (1999).
2. Aitman, T. J. et al. Copy number polymorphism in Fcgr3 predisposes to glomerulonephritis in rats and humans. [see comment]. Nature 439, 851-5 (2006).
3. Smith, J. et al. Genes expressed by both mesangial cells and bone marrow-derived cells underlie genetic susceptibility to crescentic glomerulonephritis in the rat. J Am Soc Nephrol 18, 1816-23 (2007).
4. Lai, P. C. et al. Interleukin-11 attenuates nephrotoxic nephritis in Wistar Kyoto rats. J Am Soc Nephrol 12, 2310-20 (2001).
5. Cattell, V. Macrophages in acute glomerular inflammation. Kidney Int 45, 945-52 (1994).
6. Cook, H. T. et al. Interleukin-4 ameliorates crescentic glomerulonephritis in Wistar Kyoto rats. Kidney Int 55, 1319-26 (1999).
7. Martinez, F. O., Gordon, S., Locati, M. & Mantovani, A. Transcriptional profiling of the human monocyte-to-macrophage differentiation and polarization: new molecules and patterns of gene expression. J Immunol 177, 7303-11 (2006).
8. Odegaard, J. I. et al. Macrophage-specific PPARgamma controls alternative activation and improves insulin resistance. Nature 447, 1116-20 (2007).
9. Glazier, A. M., Nadeau, J. H. & Aitman, T. J. Finding genes that underlie complex traits. Science 298, 2345-9 (2002).
10. Aitman, T. J. et al. Identification of Cd36 (Fat) as an insulin-resistance gene causing defective fatty acid and glucose metabolism in hypertensive rats. [see comment]. Nature Genetics 21, 76-83 (1999).
11. Pfarr, C. M. et al. Mouse JunD negatively regulates fibroblast growth and antagonizes transformation by ras. Cell 76, 747-60 (1994).
12. Weitzman, J. B., Fiette, L., Matsuo, K. & Yaniv, M. JunD protects cells from p53-dependent senescence and apoptosis. Mol Cell 6, 1109-19 (2000).
13. Gerald, D. et al. JunD reduces tumor angiogenesis by protecting cells from oxidative stress. Cell 118, 781-94 (2004).
14. Meixner, A., Karreth, F., Kenner, L. & Wagner, E. F. JunD regulates lymphocyte proliferation and T helper cell cytokine expression. Embo J 23, 1325-35 (2004).
15. Thepot, D. et al. Targeted disruption of the murine junD gene results in multiple defects in male reproductive function. Development 127, 143-53 (2000).
16. Gordon, S. Alternative activation of macrophages. Nat Rev Immunol 3, 23-35 (2003).
17. Mosser, D. M. The many faces of macrophage activation. J Leukoc Biol 73, 209-12 (2003).
18. Stein, M., Keshav, S., Harris, N. & Gordon, S. Interleukin 4 potently enhances murine macrophage mannose receptor activity: a marker of alternative immunologic macrophage activation. J Exp Med 176, 287-92 (1992).
19. Mantovani, A., Sica, A. & Locati, M. Macrophage polarization comes of age. Immunity 23, 344-6 (2005).
20. Rauh, M. J. et al. SHIP represses the generation of alternatively activated macrophages. Immunity 23, 361-74 (2005).
21. Chung, E. Y., Liu, J., Zhang, Y. & Ma, X. Differential expression in lupus-associated IL-10 promoter single-nucleotide polymorphisms is mediated by poly(ADP-ribose) polymerase-1. Genes Immun (2007).
22. Groux, H. & Cottrez, F. The complex role of interleukin-10 in autoimmunity. J Autoimmun 20, 281-5 (2003).
23. Llorente, L. et al. Spontaneous production of interleukin-10 by B lymphocytes and monocytes in systemic lupus erythematosus. Eur Cytokine Netw 4, 421-7 (1993).
24. Grondal, G. et al. Cytokine production, serum levels and disease activity in systemic lupus erythematosus. Clin Exp Rheumatol 18, 565-70 (2000).
25. Llorente, L. et al. Dysregulation of interleukin-10 production in relatives of patients with systemic lupus erythematosus. Arthritis Rheum 40, 1429-35 (1997).
26. Hu, X., Chen, J., Wang, L. & Ivashkiv, L. B. Crosstalk among Jak-STAT, Toll-like receptor, and ITAM-dependent pathways in macrophage activation. J Leukoc Biol 82, 237-43 (2007).
27. Ip, Y. T. & Davis, R. J. Signal transduction by the c-Jun N-terminal kinase (JNK)—from inflammation to development. Curr Opin Cell Biol 10, 205-19 (1998).
28. Davis, R. J. Signal transduction by the JNK group of MAP kinases. Cell 103, 239-52 (2000).
29. Sakurai, H. et al. Suppression of NF-kappa B and AP-1 activation by glucocorticoids in experimental glomerulonephritis in rats: molecular mechanisms of anti-nephritic action. Biochim Biophys Acta 1362, 252-62 (1997).
30. Sakurai, H. & Sugita, T. c-Jun N-terminal kinase-mediated AP-1 activation in experimental glomerulonephritis in rats. Biochem Mol Biol Int 45, 831-9 (1998).
31. Flanc, R. S. et al. A pathogenic role for JNK signaling in experimental anti-GBM glomerulonephritis. Kidney Int 72, 698-708 (2007).
32. May, R. C., Caron, E., Hall, A. & Machesky, L. M. Involvement of the Arp2/3 complex in phagocytosis mediated by FcgammaR or CR3. Nat Cell Biol 2, 246-8 (2000).
33. Clerk, A., Kemp, T. J., Zoumpoulidou, G. & Sugden, P. H. Cardiac myocyte gene expression profiling during H2O2-induced apoptosis. Physiol Genomics (2006).
34. Williams, L., Bradley, L., Smith, A. & Foxwell, B. Signal transducer and activator of transcription 3 is the dominant mediator of the anti-inflammatory effects of IL-10 in human macrophages. J Immunol 172, 567-76 (2004).
35. Tarzi, R. M., Cook, H. T., Jackson, I., Pusey, C. D. & Lord, G. M. Leptin-deficient mice are protected from accelerated nephrotoxic nephritis. Am J Pathol 164, 385-90 (2004).

1. An agent that inhibits the expression or activity of a nucleic acid molecule or polypeptide encoded by said nucleic acid molecule wherein said nucleic acid molecule or polypeptide is selected from the group consisting of:
i) a nucleic acid molecule comprising the nucleic acid sequence in FIG. 10a;
ii) a nucleic acid molecule that hybridizes under stringent hybridization conditions to the nucleic acid sequence as represented in FIG. 10a and which encodes a polypeptide that has the activity associated with Jun D;
iii) a polypeptide encoded by a nucleic acid molecule as defined in i) and ii) above;
iv) a polypeptide as represented by the amino acid sequence in FIG. 10b or 10c, wherein said agent is for use as a pharmaceutical.

2. The agent according to claim 1 wherein said agent is an inhibitory RNA.

3. The agent according to claim 2 wherein said inhibitory RNA is a small inhibitory RNA (siRNA).

4. The agent according to claim 2 wherein said inhibitory RNA molecule is at least 18 base pairs in length.

5. The agent according to claim 3 wherein said siRNA molecule is between 18 bp and 29 bp in length.

6. The agent according to claim 5 wherein said siRNA is at least one selected from the group consisting of:
(SEQ ID NO: 4)
5′- Phos/UCAGUAAAGUCUUCGUUACGCCAAA -3′
(SEQ ID NO: 5)
3′- AAAGUCAUUUCAGAAGCAAUGCGGUUU -5′
(SEQ ID NO: 6)
5′- Phos/UCCUGUUCCGUAAUCCUUGGUUCGC -3′
(SEQ ID NO: )
3′- UAAGGACAAGGCAUUAGGAACCAAGCG
(SEQ ID NO: 8)
5′- Phos/CGCAGUUCCUCUACCCUAAGGUGGC -3′
(SEQ ID NO: 9)
3′- AUGCGUCAAGGAGAUGGGAUUCCACCG -5′

7. The agent according to claim 6 wherein said siRNA is at least 2 siRNAs selected from said group.

8. The agent according to claim 6 wherein said siRNA is each one of said siRNAs.

9. The agent according to claim 1 wherein said agent is an antisense nucleic acid molecule or oligonucleotide.

10. The agent of claim 2, wherein said inhibitory RNA or said antisense nucleic acid molecule is modified.

11. The agent according to claim 1 wherein said agent is a peptide, an antibody or antibody fragment.

12. The gent according to claim 11 wherein said peptide is a modified peptide antagonist.

13. A composition comprising the agent according to claim 1.

14. The composition according to claim 13 wherein said composition is a pharmaceutical composition.

15. The composition according to claim 14 wherein said composition further includes a carrier.

16. The composition according to claim 14 wherein said composition further includes a second agent.

17. The composition according to claim 16 wherein said second agent is an anti-inflammatory agent.

18. A method to diagnose a subject suffering or having a predisposition to an inflammatory disease or condition comprising:
i) providing an isolated sample from a subject comprising a cell to be tested;
ii) determining the expression of a nucleic acid molecule or polypeptide encoded by a nucleic acid molecule comprising the nucleic acid sequence as represented in FIG. 10a or the amino acid sequence as represented in FIG. 10b or 10c;
iii) comparing the expression of said nucleic acid molecule or said polypeptide in said sample to a control sample; and
iv) determining the level of expression in said sample as an indicator of the existence or susceptibility of said subject to said inflammatory disease or condition.

19. The method according to claim 18 wherein said subject is a human.

20. The method according to claim 18 wherein said disease is an auto-immune disease.

21. The method according to claim 20 wherein said disease or condition is selected from the group consisting of: type 1 diabetes, rheumatoid arthritis, osteoarthritis, polyarthritis, gout, systemic lupus erythematosus, scleroderma, Sjorgen's syndrome, poly- and dermatomyositis, vasculitis, tendonitis, synovitis, bacterial endocarditis, osteomyelitis, psoriasis, pneumonia, fibrosing alveolitis, chronic bronchitis, chronic obstructive pulmonary disease (COPD), bronchiectasis, emphysema, silicosis, tuberculosis, ulcerative colitis and Crohn's disease, chronic inflammatory demyelinating polyradiculoneuropathy, chronic inflammatory demyelinating polyneuropathy, multiple sclerosis, Guillan-Barre Syndrome and myasthemia gravis, mastitis, laminitis, laryngitis, chronic cholecystitis, Hashimoto's thyroiditis, and including chronic inflammatory renal disease.

22. The method according to claim 18 wherein said disease is inflammatory renal disease selected from the group consisting of glomerulonephritis, crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis and tubulointerstitial nephritis.

23. The method according to claim 22 wherein said renal disease is glomerulonephritis.

24. The method according to claim 18, wherein said method includes identifying a treatment regime that would benefit said subject.

25. A method to treat a subject suffering from or predisposed to an inflammatory disease or condition comprising administering a pharmaceutically effective amount of the agent according to claim 1.

26. The method according to claim 25 wherein said subject is a human.

27. The method according to claim 25 wherein said disease is an autoimmune disease.

28. The method according to claim 27 wherein said inflammatory disease or condition is selected from the group consisting of: type 1 diabetes, rheumatoid arthritis, osteoarthritis, polyarthritis, gout, systemic lupus erythematosus, scleroderma, Sjorgen's syndrome, poly- and dermatomyositis, vasculitis, tendonitis, synovitis, bacterial endocarditis, osteomyelitis, psoriasis, pneumonia, fibrosing alveolitis, chronic bronchitis, chronic obstructive pulmonary disease (COPD), bronchiectasis, emphysema, silicosis, tuberculosis, ulcerative colitis and Crohn's disease, chronic inflammatory demyelinating polyradiculoneuropathy, chronic inflammatory demyelinating polyneuropathy, multiple sclerosis, Guillan-Barre Syndrome and myasthemia gravis, mastitis, laminitis, laryngitis, chronic cholecystitis, Hashimoto's thyroiditis, and including chronic inflammatory renal disease.

29. The method according to claim 28 wherein said disease is inflammatory renal disease selected from the group consisting of glomerulonephritis, crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis and tubulointerstitial nephritis.

30. The method according to claim 29 wherein said renal disease is glomerulonephritis.

31-35. (canceled)

36. A screening method for the identification of an agent that has JunD inhibitory activity comprising the steps of:
providing a polypeptide encoded by a nucleic acid molecule selected from the group consisting of:
a) a nucleic acid molecule comprising a nucleic acid sequence as represented in FIG. 10a;
b) a nucleic acid molecule comprising nucleic acid sequences that hybridise to the sequence identified in (a) above under stringent hybridization conditions and which encodes a polypeptide that has JunD activity;
providing at least one candidate agent to be tested;
forming a preparation that is a combination of (i) and (ii) above; and
testing the effect of said agent on the activity of JunD.

37. The method according to claim 36 wherein said polypeptide is represented by the amino acid sequence in FIG. 10b or 10c.

38. The method according to claim 36 wherein said polypeptide is expressed by a cell wherein said cell is transformed or transfected with a nucleic acid molecule that encodes a Jun D polypeptide.

39. The method according to claim 38 wherein said nucleic acid molecule is part of a vector adapted for recombinant expression of said nucleic acid molecule.

40. The method according to claim 38 wherein said cell is derived from a monocyte.

41. The method according to claim 40 wherein said cell is a macrophage.

42. The method according to claim 41 wherein said macrophage is an activated macrophage.

43. A modelling method to determine the association of an agent with a JunD polypeptide comprising the steps of:
i) providing computational means to perform a fitting operation between an agent and a polypeptide defined by the amino acid sequence in FIG. 10b or 10c; and
ii) analysing the results of said fitting operation to quantify the association between the agent and the JunD polypeptide.

44. (canceled)

45. A method to prevent organ or tissue transplantation in a subject comprising administering an effective amount of the agent according to claim 1 to prevent or inhibit organ or tissue rejection.
